(19) United States
(12) Patent Application Publication
(10) Pub. No.: US 2015/0005791 A1
(43) Pub. Date: Jan. 1, 2015

(54) ATHERECTOMY DEVICE HAVING COMBINED OPEN/CLOSE DRIVE SHAFT

(71) Applicant: Cardiovascular Systems, Inc., St. Paul, MN (US)

(72) Inventors: Victor Leo Schoenle, Greenfield, MN (US); Jeffrey A. McBroom, Champlin, MN (US)

(21) Appl. No.: 14/316,332

(22) Filed: Jun. 26, 2014

Related U.S. Application Data
(60) Provisional application No. 61/840,729, filed on Jun. 28, 2013.

(51) Int. Cl. A61B 17/3207

(52) U.S. Cl. CPC A61B 17/320758; A61B 17/32075; A61B 17/320725; A61B 2017/00367
USPC 606/159

(57) ABSTRACT

An atherectomy device may include a handle having a rotational drive mechanism therein, a drive shaft operably coupled to the drive mechanism and having an abrasive element arranged at a distal end thereof, the drive shaft comprising a first portion and a second portion, wherein the first portion comprises a spin-to-close profile and the second portion comprises a spin-to-open profile.

### CROSS-REFERENCE TO RELATED APPLICATIONS

The present application claims priority to U.S. Provisional Application No.: 61/840,729 entitled Rotating Drive Shaft with Two or More Winding Directions, filed on Jun. 28, 2013, the content of which is hereby incorporated by reference herein in its entirety. The present application is also related to U.S. Provisional Application No.: 61/613,158 entitled Drive Shaft with Improved Collapse and Column Strength, filed on Mar. 20, 2012, the content of which is hereby incorporated by reference herein in its entirety.

### FIELD OF THE INVENTION

The present application relates to devices and methods for removing tissue from body passageways, such as removal of atherosclerotic plaque from arteries, utilizing a high-speed rotational atherectomy device. More particularly, the present application relates to spindles, drive shafts, snakes, or other elongate torsion transferring elements that may be used in an atherectomy procedure. Still more particularly, the present application relates to spin-top-open and/or spin-to-close drive shafts of atherectomy devices.

### BACKGROUND

Generally, single-wound drive shafts used in Orbital Atherectomy Devices (OAD) may open or expand when loaded depending on the winding direction relative to the rotational direction of the drive shaft during operation. Such spin-to-open shafts expand under load, with the filer spacing being increased. Alternatively, or in addition, if spacing is not increased under load, the shaft length is reduced. In either case, the outer diameter of the drive shaft increases.

When the load is reduced or the shaft ceases rotating, the shaft may spring back to its static state. This spring-back action can catch or tear biological material, resulting in unintentional vessel damage and trauma. In addition, the spring-back action can dampen the force actually transmitted and applied to the abrasive element or crown attached to the drive shaft at a distal end.

In contrast, spin-to-close drive shafts may have a similar but opposite effect to that discussed above with the spin-to-open shafts. Spin-to-close shafts may wrap tighter when loaded or during rotation. In addition, the drive shaft length may be extended beyond its static length and the outer diameter may be reduced. If such a shaft is sufficiently loaded, its outer diameter may be reduced to the point that it locks onto the guide wire.

### BRIEF SUMMARY OF THE INVENTION

The present application, in some embodiments, relates to an atherectomy device including a handle having a rotational drive mechanism therein and a drive shaft operably coupled to the drive mechanism and having an abrasive element arranged at a distal end thereof. The drive shaft may include a first portion and a second portion and the first portion may include a spin-to-close profile and the second portion may include a spin-to-open profile. In some embodiments, the drive shaft further comprises a third portion arranged between the first and second portion. The first portion may include a proximal portion of the drive shaft and the second portion may include a distal portion of the drive shaft. In some embodiments, the third portion may include a spin-to-open profile. In additional embodiments, the device may also include a sheath having a lumen extending therethrough and a portion of the drive shaft may be arranged within the lumen. In some embodiments, the lumen may include a diameter defined by an inner wall of the sheath and the spin-to-open profile of the third portion may be configured to contact the inner wall when operating the drive shaft.

The figures and the detailed description which follow more particularly exemplify these and other embodiments of the invention.

### BRIEF DESCRIPTION OF THE DRAWINGS

The invention may be more completely understood in consideration of the following detailed description of various embodiments of the invention in connection with the accompanying drawings, which are as follows.

FIG. 1 is a perspective view of an atherectomy device, according to some embodiments.

FIG. 2 is a close-up and partial cutaway view of a portion of the distal end of the atherectomy device of FIG. 1 showing the drive shaft portion of the device arranged within the catheter or sheath.

FIG. 3 shows a drive shaft having a spin-to-close profile for a clockwise driven drive shaft, according to some embodiments.

FIG. 4 shows a drive shaft having a spin-to-open profile for a clockwise driven drive shaft, according to some embodiments.

FIG. 5 shows a combined, spin-to-open/spin-to-close drive shaft, according to some embodiments.

FIG. 6 shows a coupling element for coupling a spin-to-open and a spin-to-close drive shaft, according to some embodiments.

FIG. 7 shows a combined, spin-to-open/spin-to-close drive shaft, according to some embodiments.

### DETAILED DESCRIPTION

While the invention is amenable to various modifications and alternative forms, specifics thereof are shown by way of example in the drawings and described in detail herein. It should be understood, however, that the intention is not to limit the invention to the particular embodiments described. On the contrary, the intention is to cover all modifications, equivalents, and alternatives falling within the spirit and scope of the invention.

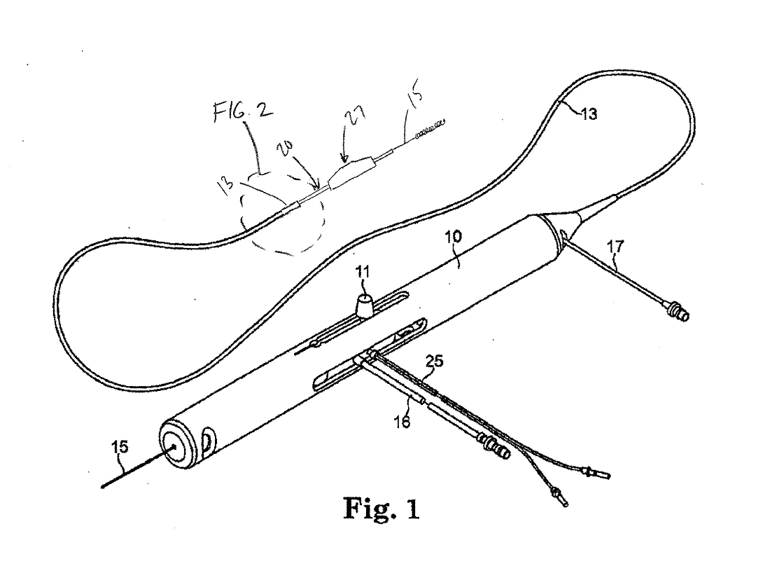

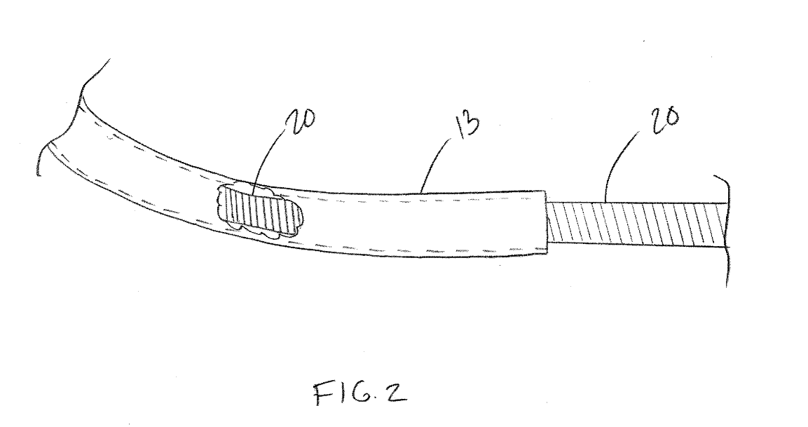

FIG. 1 illustrates one embodiment of a rotational atherectomy device. As shown, the device may include a handle portion 10, an elongated, flexible drive shaft 20 having a head 27 in the form of a burr, crown, or bit, and an elongated catheter or sheath 13 extending distally from the handle portion 10. The drive shaft 20 may be constructed from helically coiled wire and the head 27 may be attached thereto. As shown in FIG. 1 and close-up view in FIG. 2, the catheter or sheath 13 may include a lumen in which most of the length of the drive shaft 20 is disposed, except for the head 27 and, in some cases, a short section distal to the head 27. The drive shaft 20 also may contain an inner lumen, permitting the drive shaft 20 to be advanced and rotated over a guide wire 15. A fluid supply line 17 may be provided for introducing a cooling and lubricating solution (typically saline or another biocompatible fluid) into the catheter 13.

The handle 10 may include a turbine (or similar rotational drive mechanism) for rotating the drive shaft 20 at high speeds. The handle 10 typically may be connected to a power source, such as compressed air delivered through a tube 16 or electrical power delivered with an electrical connection. A pair of fiber optic cables 25, or a single fiber optic cable, for example, may also be provided for monitoring the speed of rotation of the turbine and drive shaft 20. The handle 10 also may include a control knob 11 for advancing and retracting the turbine and drive shaft 20 with respect to the catheter 13 and the body of the handle.

As will be appreciated, when the rotational drive mechanism in the handle 10 is actuated and the drive shaft is spun or otherwise rotated, the drive shaft 20 may experience resistance to rotation at the distal end of the drive shaft 20 and/or along its length. The torsional force within the drive shaft 20 may be lesser or greater depending on the amount of resistance experienced by the drive shaft 20 and the amount of rotational force imparted by the drive mechanism. In some cases, if the distal end encounters an obstruction and comes to an abrupt stop or experiences a quick acting resistance, the torsion in the shaft 20 may be affected by the rotational momentum of the drive shaft 20 and/or drive mechanism as well.

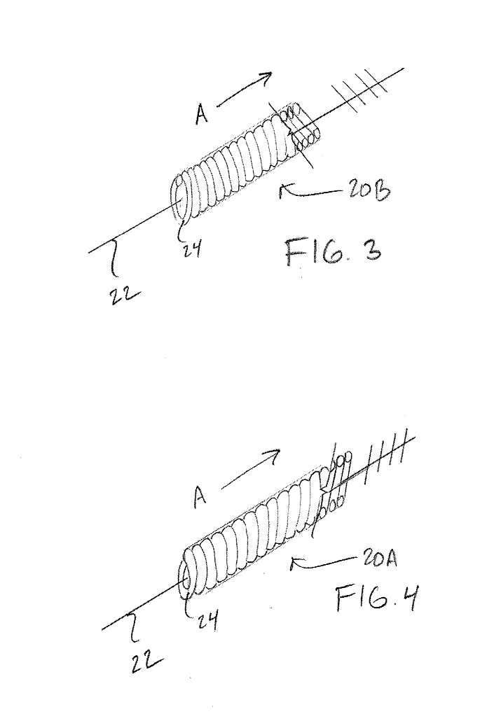

It is to be appreciated that the direction that a drive shaft 20 is wound, compared to the direction it is driven, affects its response to being driven and, in particular, its response to startup and its response to encountering an obstruction. That is, a drive shaft 20 may include a coiled wire that may be formed by winding on a mandrel, or otherwise formed. As shown in FIGS. 3 and 4, the drive shaft 20 may be wound in one of two directions about a longitudinal axis as the coil extends along the axis in a direction A. As shown in FIG. 3, the wire 24 may be wound or coiled counterclockwise about the axis 22. In contrast, as shown in FIG. 4, the wire 24 may be wound or coiled clockwise about the axis 22. Depending on the direction that the resulting drive shafts 20 are driven, the coiled wires may define a spin-to-open drive shaft 20A or a spin-to-close drive shaft 20B. For purposes of discussion and simplicity, the present discussion generally assumes that the driven direction is a clockwise drive direction when viewing a device from a proximal, or driving or handle, end of the device. For purposes of FIGS. 3 and 4, the drive end is at the left of the figure and resistance may be present along the coil and at the right side of the figure. As such, referring again to FIG. 3, this type of drive shaft 20 may be termed a spin-to-close drive shaft. This is because as the drive shaft 20B spins in the clockwise direction, resistance to such spinning has a tendency to cause the coil to get tighter or close. In contrast, as shown in FIG. 4, this type of drive shaft 20 may be termed a spin-to-open drive shaft 20A. This is because as the drive shaft 20A spins in the clockwise direction, resistance to such spinning has a tendency to cause the coil to get looser or open.

As shown near the distal end of each example in FIGS. 3 and 4, a portion is shown in cross-section showing that the windings or filers have a pitch defined by how much the windings or filers lay over relative to a line perpendicular to the longitudinal axis 22. For purposes of discussion going forward, schematic drawing lines that cross the longitudinal axis that are tipped rearwardly or, close to perpendicular, as in FIG. 3, will be understood to reflect a spin-to-close drive shaft 20B and lines that cross the longitudinal axis that are tipped forwardly as in FIG. 4, will be understood to reflect a spin-to-open drive shaft 20A. However, it is to be appreciated that these definitions are with respect to a clockwise drive direction and it is understood that reversing the drive direction may change a spin-to-open drive shaft 20A to a spin-to-close drive shaft 20B and vice versa.

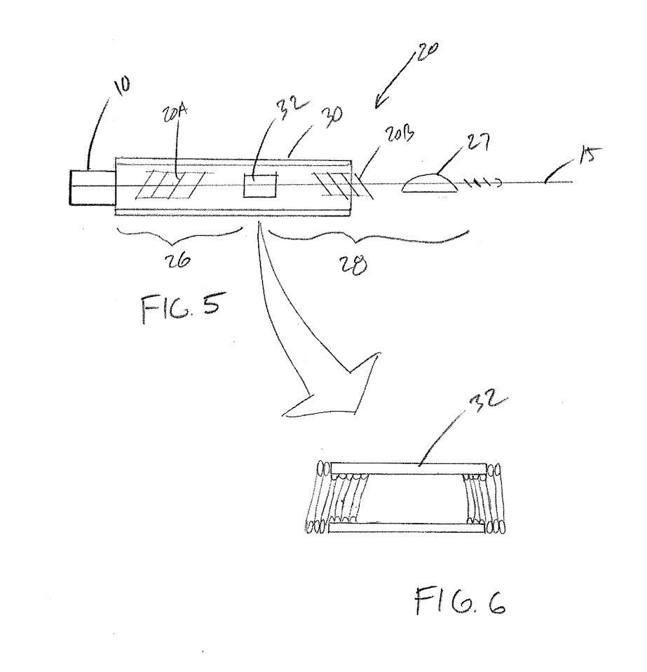

Referring now to FIG. 5, and contemplating the difficulties and issues associated with spin-to-open 20A and spin-to-close 20B drive shafts, a drive shaft 20 with a combination of spin-to-open 20A and spin-to-close 20B elements is shown. As mentioned previously, an atherectomy device may include a handle portion 10 having an air-pressure driven turbine, an electric motor, or another type of drive mechanism for rotationally driving the drive shaft 20. The drive shaft 20 may extend distally from the handle 10 to a distal end where a crown 27 may be positioned. The crown 27 may be usable to clear arterial blockages and the like. In the present embodiment, the drive shaft 20 may include a proximal portion 26 having a spin-to-open profile 20A and a distal portion 28 having a spin-to-close profile 20B. The proximal portion 26 and some of the distal portion 28 may be slidable and rotatable within a catheter or sheath 30 and the proximal portion 26 and distal portion 28 may be secured to one another with a coupling element 32.

When the present drive shaft 20 is loaded, the proximal spin-to-open section 20A may open within the sheath 30 and the relationship of the drive shaft 20 being within the sheath 30 may be advantageous for at least two reasons. First, the sheath 30 may protect against the outwardly expanding filers encountering biological material (i.e., arterial wall) that may be captured when the expanded filers relax, further limiting trauma potential. Secondly, the sheath 30 may limit the shaft diameter expansion at the proximal spin-to-open section 20A allowing the size of the drive shaft 20 to be controlled.

In contrast, the distal spin-to-close portion 20B, when loaded, may close or shrink in diameter. However, the rigidity of the spin-to-close portion 20B relative to the spin-to-open portion 20A may be slightly higher such that the spin-to-close portion 20B may maintain its static diameter and refrain from locking onto the guide wire 15. As such, the drive shaft 20 may maintain its maneuverability both longitudinally and rotationally relative to the guide wire 15.

The combination of a spin-to-open 20A and spin-to-close 20B drive shaft may reduce or eliminate changes in overall drive shaft 20 length because shortened length in one portion of the drive shaft 20 may be compensated for by elongated length in another portion. The relative stiffness of the portions and the relative lengths of each portion may be selected to provide a length change balance between the two portions. Where the relative length change of each portion is substantially opposite and of the same magnitude, the distal end of the drive shaft 20 may maintain its position before, during, and after startup and when encountering an obstruction. As such, the abrasive element or crown 27 attached to a distal portion of the drive shaft 20 may be less likely or unlikely to jump or spring back (or forward) when loaded. Varying the lengths and filer diameters in certain embodiments of the present invention may allow for engineered and customized responses from a loaded drive shaft 20.

It is to be appreciated that while a two-part spin-to-open 20A and spin-to-close 20B system has been described, still other numbers of portions and combinations of spin-to-open 20A and spin-to-close 20B portions may be provided. For example, more than one spin-to-open 20A section and/or more than one spin-to-close 20B section, each section having a combination of fixed or varying filer diameters, counts and shaft diameters may be provided.

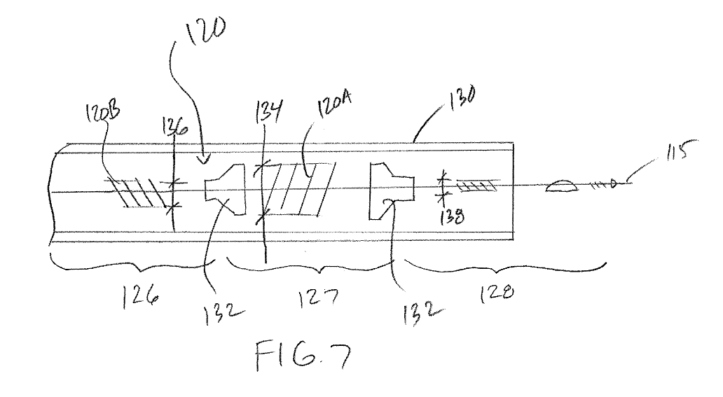

As shown in FIG. 6, the proximal and distal portions 26, 28 of the drive shaft 20 may be secured to one another with a coupling element 32. The coupling element 32 may include a sleeve type coupling element 32 such as a hypotube coupler with an inner diameter smaller than the outer diameter of the drive shaft 20 such that a portion of the drive shaft 20 may be turned down, step ground, or otherwise reduced in diameter and placed within the coupling element 32 for a friction fit. In other embodiments, in addition or in alternative to a friction fit, a welded, brazed, adhered, or other connection to the coupling element 32 may be provided. In still other embodiments, two adjoining drive shafts 26, 28 may be sleeved, one within the other, for a friction fit and/or welded, brazed, or adhered, for example. Still other coupling type connections such as those used to secure a crown 27 to the drive shaft 20 may be provided. For example, coupling connections and method such as those described in U.S. patent application Ser. No. 14/041,559 entitled Method of Attaching and Element to a Driveshaft filed on Sep. 30, 2013 may be provided. The contents of U.S. patent application Ser. No. 14/041,559 are hereby incorporated by reference herein in their entirety. The coupling element 32 arranged between one or more drive shaft portions 26, 28 may be relatively rigid or a more flexible coupling element 32 may be provided.

Referring now to FIG. 7, multiple different features of particular portions of a drive shaft 120 may be selected such that the drive shaft 120 as a whole may perform in a particular manner. For example, as shown, a relatively stiff proximal portion 126 may be provided followed by a central portion 127 having a relatively flexible torsional stiffness and having a spin-to-open profile 120A. Yet another portion 128 of the drive shaft may include an additional relatively flexible portion with a smaller diameter and having a spin-to-close 120B or a spin-to-open 120A profile. The atherectomy crown 127 may be positioned on this latter portion 128 which may be adapted to extend out of the distal end of the sheath 130 for purposes of addressing an occlusion or other obstruction.

In the particular embodiment shown, the proximal portion 126 may, for example, include a multiple filer drive shaft such as a 6-filer section. Other numbers of filers including 5, 4, 3, 2, or 1 or numbers higher than 6 may also be used. This portion 126 of the drive shaft 120 may be a spin-to-close drive shaft 120B, but its stiffness may be such that it refrains from locking down onto the guide wire 115 when activated or driven.

The central portion 127 may include a relatively flexible spin-to-open profile 120A. The spin-to-open profile 120A may have a size and stiffness that is selected such that when actuated, the central portion 127 opens up and contacts the inside of the sheath 130. In some embodiments, as shown, the at rest diameter 134 of the central portion 127 may be slightly larger than the diameter 136 of the proximal portion 126.

The distal portion 128 may include a relatively flexible spin-to-open 120A or spin-to-close 120B profile. For example, the distal portion 128 may include a soft flexible tip portion including a 3-filer tip with a crown 127 positioned thereon. In some embodiments, as shown, the distal portion 128 may have a diameter 138 smaller than that of the central portion 127 and may also have a diameter 138 smaller than that of the proximal portion 126.

As shown, the varying diameters of the several portions 126, 127, and 128 may be accommodated by providing coupling elements 132 in the form of reducers to transition from larger diameter portions to smaller diameter portions. In some embodiments, the outside of the reducers may be tapered as shown or a constant outside diameter with varying inside diameters may be provided. Still other approaches to coupling may be provided as discussed above.

Several advantages may result from the combinations of different drive shaft properties along the length of the overall drive shaft. For example, the multi-direction winding design may provide reduced crown movement at startup. In addition, the multi-direction winding design may provide improved saline flow at higher rotational speeds. This is because spinning large diameter wires in a suitable direction may cause the wires to act as pump vanes functioning to advance the saline flow along the device within the sheath. Multi-direction winding provides the ability to tailor how the driveshaft reacts to overloading by allowing the driveshaft to expand and contact the saline sheath or to contract and contact the guide wire or a combination of both in different sections. Multi-direction winding design may provide different section properties to change stiffness or flexibility and, as such, affect the performance of a particular portion of the drive shaft and the overall performance. In some embodiments, several different filer cross-sectional shapes and areas may be used such as round, flat with edge radius, square, rectangle, etc. The areas may be adjusted by using heavy wire or fine wire or some variance in the thickness of the wire. The several different filer cross-sectional shapes and areas may be selected to provide a particular desired performance. In addition, as discussed with respect to FIG. 7, different number of filers can be used to change mechanical properties in each section of a drive shaft. In addition to varying the cross-sectional shape or area from one section of the drive shaft to another, the cross-sectional area may be varied within a single section by grinding, etching, stretching, or any other process known in the art to reduce or change the wire cross-sectional area or shape.

It is to be appreciated that several factors have been discussed with respect to each portion of the drive shaft including spin-to-open, spin-to-close, cross-sectional shape and area, diameter, material, and variations of any of these within a particular portion. Selecting from these several factors may provide a drive shaft designer with a high level of flexibility to create a drive shaft with properties and/or performance characteristics never before seen or appreciated. With respect to any given property, the present application may include one of several combinations of arrangement potentially depending on the number of portions. For example, with respect to spin-to-open (SPO) and spin-to-close (SPC), if there are two portions of the drive shaft, there may be approximately four different arrangements that could be used from the proximal end to the distal end as follows:
a. SPO; SPO
b. SPO; SPC
c. SPC; SPO
d. SPC; SPC

As the number of portions of the drive shaft increases, all combinations of SPO and SPC shall be considered to be within the scope of the present disclosure. For example, where three portions are provided, the following arrangements may be provided:
a. SPO; SPO; SPO
b. SPO; Spa; SPC
c. SPO; SPC; SPO
d. SPO; SPC; SPC
e. SPC; SPO; SPO
f. SPC; SPO; SPC
g. SPC; SPC; SPO
h. SPC; SPC; SPC

Any number of portions may be provided and any combination of properties for the portions may be used. In cases where there is no change in the spin-to-open or spin-to-close profile from one section to another, there may be changes in other properties such as cross-sectional shape or area, diameter, or material, for example. With respect to the other properties and factors discussed, all combinations may also be provided.

The present invention should not be considered limited to the particular examples described above, but rather should be understood to cover all aspects of the invention. Various modifications, equivalent processes, as well as numerous structures to which the present invention may be applicable will be readily apparent to those of skill in the art to which the present invention is directed upon review of the present specification.

What is claimed is:

1. An atherectomy device, comprising:
a handle having a rotational drive mechanism therein; and
a drive shaft operably coupled to the drive mechanism and having an abrasive element arranged at a distal end thereof, the drive shaft comprising a first portion and a second portion, wherein the first portion comprises a spin-to-close profile and the second portion comprises a spin-to-open profile.

2. The device of claim 1, further comprising a sheath having a lumen extending therethrough, wherein a portion of the drive shaft is arranged within the lumen.

3. The device of claim 2, wherein the lumen comprises a diameter defined by an inner wall of the sheath and the spin-to-open profile of the first portion is configured to contact the inner wall when operating the drive shaft.

4. The device of claim 1, wherein the first portion forms a proximal portion of the drive shaft and the second portion forms a distal portion of the drive shaft with the abrasive element on a distal end thereof.

5. The device of claim 4, wherein the first portion is secured to the second portion with a coupling element.

6. The device of claim 5, further comprising a sheath having a lumen extending therethrough, wherein the first portion of the drive shaft is arranged within the lumen and the distal end of the second portion extends out of a distal end of the sheath.

7. The device of claim 1, wherein the first portion forms a distal portion of the drive shaft and the second portion forms a proximal portion of the drive shaft with the abrasive element on a distal end thereof.

8. The device of claim 1, wherein the drive shaft further comprises a third portion arranged between the first and second portion.

9. The device of claim 8, wherein the first portion comprises a proximal portion of the drive shaft and the second portion comprises a distal portion of the drive shaft.

10. The device of claim 9, wherein the third portion comprises a spin-to-open profile.

11. The device of claim 10, further comprising a sheath having a lumen extending therethrough, wherein a portion of the drive shaft is arranged within the lumen.

12. The device of claim 11, wherein the lumen comprises a diameter defined by an inner wall of the sheath and the spin-to-open profile of the third portion is configured to contact the inner wall when operating the drive shaft.

13. The device of claim 12, wherein the first portion comprises a relatively stiff drive shaft.

14. The device of claim 13, wherein the first portion comprises a multiple filer section.

15. The device of claim 14, wherein the first portion comprises six filers.

16. The device of claim 13, wherein the second portion comprises one of a spin-to-open profile and a spin-to-close profile and the second portion comprises a relatively flexible drive shaft.

17. The device of claim 16, wherein the abrasive element is arranged on the second portion and the second portion is configured for extending out of a distal end of the sheath to expose the abrasive element.

18. The device of claim 12, wherein the first portion has a first diameter, the second portion has a second diameter smaller than the first diameter, and the third portion has a third diameter larger than the second diameter.

19. The device of claim 18, wherein the third diameter is larger than the first diameter.

20. The device of claim 8, wherein each of the first, second and third portion comprise filer cross-sections comprising one of round, flat with edge radius, square, and rectangle.
